(19) United States
(12) Patent Application Publication
(10) Pub. No.: US 2006/0148679 A1
(43) Pub. Date: Jul. 6, 2006

(54) Glycosolated enkephalin agents

(76) Inventors: Robin L Polt, Tucson, AZ (US); Edward J. Nilsky, Biddeford Pool, ME (US)

Correspondence Address: QUARLES & BRADY LLP, FIRSTAR PLAZA, ONE SOUTH PINCKNEY STREET, P.O BOX 2113 SUITE 600, MADISON, WI, 53701-2113, US

(21) Appl. No.: 10/540,443

(22) PCT Filed: Feb. 24, 2004
(86) PCT No.: PCT/US04/05340

§ 371 Date: Jun. 22, 2005

Related U.S. Application Data
(60) Provisional application No. 60/449,989, filed on Feb. 25, 2003.

(51) Int. Cl. A61K 38/33

(52) U.S. Cl. 514/8

(57) ABSTRACT

It is taught here that enkephalin peptides which are glycosylated with a disaccharide will be transported across the blood brain barrier and deliver analgesic effects superior to morphine when introduced into the bloodstream. Glycosylated peptides with disaccharides attached are superior to analogous peptides with either mono- or tri-saccharides attached.

### CROSS-REFERENCE TO RELATED APPLICATIONS

This application claims priority from U.S. provisional patent application Ser. No. 60/449,989, filed Feb. 25, 2003.

### STATEMENT REGARDING FEDERALLY SPONSORED RESEARCH OR DEVELOPMENT

This invention was made with United States government support awarded by the following agencies: Office Naval Research, Contract No. N00012-02-1-0471 and National Science Foundation, Contract No. 9526909. The United States has certain rights in this invention.

### BACKGROUND OF THE INVENTION

Throughout the history of human medicine, various compounds have been used for the relief of pain. In particular, a class of compounds of plant origin known as opiates have been used since prehistoric periods for analgesic and euphoric purposes. Even today the opiate drug morphine is used as an analgesic for significant pain, and morphine is still an important benchmark for clinical studies. Morphine is the most widely prescribed injectable opioid today, despite its narcotic side effects. Acute opioid toxicity from overdose can result in respiratory depression and death, whereas chronic use can let to physical dependence, addiction, and sever constipation.

Endogenous opioid peptides are synthesized vertebrates in general, and mammals in particular, and bind to the same receptors as the exogenous opioid molecules including morphine. The endogenous peptides are known by the generic term endorphins, and endorphins have been subject of much discussion and research since their discovery in the 1970s. Endorphins are believed to be the natural source of various euphoric experiences reported by people, including the “runner's high” and the feelings experienced by some after eating chocolate. Although the evidence about these experiences is to a large degree subjective, there is no question that endogenous endorphin production plays a critical role in the various sensory emotional motivational and cognitive functions.

One class of endorphin is known as the enkephalins. Enkephalins are small peptides that engage the opioid receptors with high specificity. There are both natural and synthetic enkephalins. Enkephalins are well known to actively engage the opioid receptors and can produce strong analgesic effects when delivered to the brain. However, the use of endorphins in general, or enkephalins in particular, has not moved from the theoretical to the therapeutic reality, in large part based on difficulties in their administration and stability, and an inability to deliver the molecules through the blood brain barrier.

The blood-brain barrier is the barrier that exists between the mammalian blood stream and the cerebrospinal cavity. Some small opioid molecules, such as morphine, delivered to the blood stream are capable of passing into the brain. In general, peptides such as endorphins introduced into the human blood stream do not pass the blood-brain barrier. As such, the experience has been that enkephalin molecules, whether synthetic or native, when delivered by intravenous injection into mammalian models have been found disappointing in their delivery of analgesic effect to the subject.

The blood-brain barrier has two major components. The endothelial layer lies between the arterial blood and the brain capillaries and the interstitial fluid of the brain. The epithelial layer lies between the venous blood and the cerebral spinal fluid in the choroid plexus. In the spinal cord, the blood brain barrier only consists of the endothelial barrier. The blood-brain barrier represents not only a physical obstacle to the passage of molecules, but a metabolic one as well, since the layers possess both oxidative enzymes and peptidases which can degrade metabolically unstable substances, such as peptides, before they can reach the cerebral spinal fluid. The enzymatic barriers may be an important part of the barrier created by the blood-brain barrier in excluding peptide pharmaceuticals from the central nervous system. It should also be noted that delivery of molecules to the cerebral spinal fluid also does not guarantee that the drug will enter the brain, as many molecules are rapidly exported back to the blood stream by active processes, even if delivered to the cerebral spinal fluid. The transport of drugs across the blood brain barrier falls into two broad categories, passive diffusion and facilitated diffusion which is mediated through a specific transport mechanism. Since peptides are relatively large and relatively hydrophilic, they do not cross the blood brain barrier by passive diffusion, and they do require either facilitated diffusion or active transport mechanisms. Various invasive drug delivery strategies have been used to deliver drugs behind the blood-brain barrier including intracerebral fusions or intrathecal implants. While there are medical situations where such invasive techniques are justified in humans, it is clear that non-invasive methodologies have the potential for the much wider therapeutic application of for peptide-based pharmacotherapy in general and opioid-peptide based analgesics in particular.

### BRIEF SUMMARY OF THE INVENTION

The present invention is summarized as a method for delivering analgesia to an individual by administering to the bloodstream of the individual an effective amount of an analgesic molecule which is a glycosylated enkephalin, the glycosylation being a disaccharide sugar moiety.

The present invention is also summarized in a therapeutic agent intended for delivery to patients wherein the agent is a glycosylated enkephalin, the glycosylation being a disaccharide.

Other object advantages and features of the present invention will be apparent from the following specification.

### BRIEF DESCRIPTION OF THE SEVERAL VIEWS OF THE DRAWINGS

FIG. 1 is a graphical representation of data from the experiments described below illustrating the effect of exemplary glycosylated enkephalins delivered to the brain.

FIG. 2 is a graphical representation of data from the experiments described below illustrating the effect of exemplary glycosylated enkephalins delivered to the bloodstream.

FIG. 3 is a graphical representation of data from experiments showing opioid receptor binding and analgesic effectiveness of exemplary glycosylated enkephalins.

FIG. 4 is a graphical representation to illustrate the differences in effectiveness between delivery to the brain and delivery to the blood for exemplary glycosylated enkephalins.

### DETAILED DESCRIPTION OF THE INVENTION

It has previously been suggested that adding sugars to enkephalins can add to the structural stability of the enkephalin molecule. It has also been shown that glycosylated enkephalins show appreciable, yet weakly saturable transport, across the blood-brain barrier, and that the molecules which do transport bind strongly to opioid receptors in the brain. Here it is reported that a class of glycosylated enkephalins, those enkephalins which have a disaccharide sugar attached to them, are more efficiently transported across the blood-brain barrier, and thus are more effective at delivering analgesic effects that either monosaccharide or trisaccharide glycosylated enkephalins. It has also been discovered that these same compounds produce potent antinociceptive effects in a variety of animals models and may have beneficial anti-depressive properties as well. These findings will make practical the use of enkephalins as effective analgesic agents delivered to the bloodstream.

The exact mechanism which explains why disaccharide glycosylated enkephalins are superior to other glycosylated enkephalins, in uncertain, but it is believed that the explanation is related to the hydrophobic/hydrophilic balance of the molecules. Hydrophobic peptides, such as non-glycosylated enkephalins, insert into biological membranes where they are rapidly degraded by peptidases, and are thus not highly susceptible to the diverse transport mechanisms found in all cellular membranes. While the addition of a single sugar to an enkephalin increases the hydrophilicity of the molecule, and aids in transport across the blood-brain barrier to some extent, the addition of a disaccharide sugar to the enkephalin is much better. Interestingly, when a tri-saccharide sugar is added to an enkephalin, it is less effective than a similar enkephalin with a disaccharide attached, presumably due to an imbalanced amphipathicity. It is also believed that an amphipathic molecule is also the most effective, i.e. a molecule which has both hydrophobic and hydrophilic regions. If a glycopeptide molecule spends too much time in the aqueous phase, there will not be enough interaction with the membrane in order to undergo transcytosis (endocytosis on the blood side, followed by exocytosis on the brain side). Conversely, if a glycopeptide spends too much time associated with the membrane, it will not escape the membrane before it is degraded by peptidases. An enkephalin with a single disaccharide attached to its address segment, as discussed below, is more effective than a similar enkephalin with two monosaccharides attached to it in different sites. It is theorized that having both polar and non-polar domains allows the molecules to both pass the blood-brain barrier and bind effectively to the opioid receptors, and a single disaccharide attachment seems to provide the proper balance of these domains for the purposes of transport of an enkephalin across the blood brain barrier by transcytosis. Whether or not this theory is correct in all detail, it is clear that for glycopeptide enkephalins, molecules with disaccharides attached are much more easily passed through the blood-brain barrier.

Enkephalin and endorphin peptides may be thought of as having both an message segment and an address segment. The message segment is portion of the molecule that binds to the receptor and is quite small, typically being the four amino acid motif YGGF in native enkephalins. The address portion appears to control membrane binding and may serve to help modify receptor specificity. As is well known, there are several class of opioid receptors, with the three accepted subtypes being known as by the classifications mu (μ), delta (δ), and kappa (κ), with the corresponding clones receptors MOR, DOR and KOR. It is known that various endorphins and enkephalins bind preferentially to different classes of receptors. A listing of some endorphins and enkephalins (with single letter amino acid designations) and the receptors to which they bind is presented as Table 1 below. The message segments are underlined.

TABLE 1
Naturally Occurring Opioid Peptide Sequences.

| | | |
|---|---|---|
| Peptide | Sequence | Subtype |

| | | |
|---|---|---|
| Met-Enkephalin | YGGFM | μ/δ |
| Leu-Enkephalin | YGGFL | δ/μ |
| Dynorphin A | YGGFLRRIRPKLKWNNQ | κ(μ) |
| Dynorphin B | YGGFLRRQFKVVT | κ(μ,δ) |
| α-Neoendorphin | YGGFLRKY | κ(μ,δ) |
| β-Neoendorphin | YGGFLRKYP | κ(μ,δ) |
| β_{h-Endorphin} | YGGFMTSEKSQTPLVTLFKNAII | μ/δ |
| | KNAYKKGE | |
| Peptide E | YGGFMRRVGRPEWWMDYQKR | μ/κ |
| | YGGFL | |
| Peptide F | GGEVLGKRYGGFM | — |
| Nociceptin | FGGFLRRIRPKLKWNNQ | ORL |
| Deltorphin | YmFHLMD-CONH₂ | δ |
| Dermorphins | YaFGYPS-CONH₂ | μ |
| Morphiceptin | YPFP-CONH₂ | μ |
| β-Casomorphin | YPFPGPI | μ |
| Endomorphin-1 | YPWF-CONH₂ | μ |
| Endomorphin-2 | YPFF-CONH₂ | μ |
| Rubiscolin-6 | YPLDLF | δ |

The classic motif for opioid receptor binding is the YGGF sequence. While some variations are possible in this motif, it appears that the first tyrosine and the fourth phenylalanine are invariant requirements of enkephalins. The discovery of natural opioid peptides in the skin of the frog Phyllomedusa bicolor, which naturally produces the enantiomeric D-amino acids, led to investigations of other D-amino acids which can substitute for the glycine intermediate residues in the motif. In particular, the several motifs with a D-amino acids, including Tyr-D-Cys-Gly-Phe, Tyr-D-Ala-Gly-Phe, and Tyr-D-Thr-Gly-Phe have been found effective synthetic enkephalin message sequences. Synthetic enkephalin analogues with a D-amino acid substituted for the first glycine have been designed to bias the conformation of the molecule to obtain greater affinity for opioid receptors. Note that in the Table 1 above and 2 below that the small case letter designation refers to a D-amino acid, such as “t” referring to D-Thr.

It is taught here that the addition of the disaccharide to the enkephalins of the present invention is most effective when the disaccharide is attached to the address portion of the peptide opioid molecule. The addition of the sugar moiety to the address portion of the molecule seems to assist in transport across the blood barrier without preventing either delivery of the molecule to the receptor or binding to the receptor. Additions of sugar groups to the message portions of the molecule are less effective in producing antinociception by introduction of the molecule to the bloodstream.

The transport segment of the molecules described here is a disaccharide moiety. It is taught here that disaccharides are the superior sugar for the transport of enkephalins across the blood-brain barrier as the proper amphipathic balance to the enkephalins. Suitable disaccharides include all of the normal native disaccharides, including but not limited to sucrose, trehalose, saccharose, maltose, lactose, cellobiose, gentibiose, isomaltose, melibiose, and primeveose. For each particular enkephalin, the most suitable disaccharide can be determined by empirical experimentation.

The glycosylated enkephalins described here can be made by a number of techniques. The synthesis of small peptides by solid phase peptide synthesis is now a well understood and reproducible general process. Many resins are commercially available and suitable for this synthesis including Wang, Pal, Rink amide, Rink acid and Sasrin resins. Small peptides can also be produced by protein expression systems in microbial hosts or produced by in vitro cell free peptide synthesis. In addition to the natural endorphins and enkephalins listed in Table 1 above, a large number of related synthetic endorphins and enkephalins are also known. Some synthetic enkephalins are listed in Table 2 below. Any of the numerous small peptide enkephalin molecules can be made by these or other methods for use within the present invention.

Suitable methods for the linkage of sugars to small peptides are also known. It is preferred that the sugars be linked to the peptides by an O-linkage to a side chain of a peptide in the address segment of the peptide. An O-linkage means that the sugar is linked to the hydroxyl side chain of an amino acid. U.S. Pat. No. 5,727,254 describes useful methods to add sugars by an O-linkage to natural or synthetic peptides or amino acids. While it is not critical whether the disaccharide moiety is first attached to an amino acid which is then incorporated into a peptide enkephalin or if the amino acids are first assembled into an enkephalin which is then glycosylated, the usual practice has been to glycosylate a serine or threonine amino acid and then incorporate that glycosylated amino acid into the solid phase synthesis of the opioid peptide.

The ability of glycosylated enkephalins to efficiently transport across the blood-brain barrier may be evaluated by comparing the results of the administration of those molecules into the cerebrospinal space against similar results from intravenous administration of the same molecule. It has been found that glycosylated enkephalins with either mono- or tri-saccharides attached will deliver analgesia when delivered to the brain and will transport to some degree across the blood brain barrier. However, these molecules will not transmit efficiently across the blood-brain barrier. By contrast, glycosylated enkephalins with disaccharides attached to them will deliver effective analgesia when delivered to the brain or delivered to the bloodstream. As described below, some of these molecules deliver analgesic effect which is a multiple of the effects of morphine.

It is anticipated that the glycosylated enkephalins of the present invention will prove useful clinical drugs for analgesia and anti-depression. For clinical use, the glycosylated peptides would be made in suspension and packaged and labeled with suitable instructions for use with patients. The drugs could be delivered intravenously and still bind to the appropriate receptors in the brain, due to the passage of the molecules through the blood-brain barrier. Other adjuvants, additives and potentiating factors might also be includes in such formulations.

### EXAMPLES

Glycopeptides have been synthesized in a variety of peptide sequences and with a variety of sugars attached. The following is a typical glycopeptide assembly and synthesis protocol.

The 6-residue peptide and glycopeptides were manually synthesized using modified solid-phase FMOC chemistry with HBTU/HOBt promoted peptide coupling (2.0 eq./2.0 eq. per 1.5 eq. of amino acid). Coupling reaction times varied from 40 to 90 minutes, and were monitored by the Kaiser ninhydrin test. The —OAc protecting groups were removed from the carbohydrate with H₂NNH₂.H₂O, and —OC(CH₃)₃ side chain protecting groups were cleaved with 90% F₃CCOOH in CH₂Cl₂, which also effected cleavage from the resin. The crude peptide were precipitated with ice-cold ether, filtered, dissolved in water and lyophilized. Purification was carried out on a Perkin-Elmer LC250 HPLC using a preparative-scale (700×45 mm) Vydac C₁₈ reverse-phase peptide chromatography column. The following conditions were used: a linear AB gradient of CH₃CN/0.1% aq. F₃CCOOH moving from 10%-50% CH₃CN over 30 min. at a flow rate of 7 mL/min at RT. After preparative HPLC, all fractions were analyzed by analytical HPLC for purity, using a Hewlett-Packard Series II 1040 analytical HPLC, with a linear AB gradient of CH₃CN/0.1% aq. F₃CCOOH moving from 10%-40% CH₃CN over 40 min. at a flow rate of 1 mL/min at RT. Water used for HPLC purification was triple-filtered, and degassed with argon for two hours prior to use. HPLC grade CH₃CN was purchased from Fischer Scientific.

The mouse anti-nociception tests were conducted with male ICR mice weighing 25-35 grams using the 55° C. tail flick test. A baseline latency was taken for each mouse. Then the mice were injected with drug and tested for antinociception at various times post-injection. A ten second cut off point was used to avoid tissue damage. The percent of antinociception was calculated as ((Test latency-Control Latency)/(10-Control Latency))×100. The majority of the drugs were tested at varying dosages.

All of the glycopeptides listed in the following Table 2 have been synthesized. Note the in the nomenclature used in Table 2, the D-threonine residues are indicated at “t,” the “L-Ser” indicate the isomer of serine and not a second leucine, and the sugars are indicated in parenthesis at their site of attachment. The molecules have been tested for anti-nociception in the rat animal model using the standard tail flick assay. The molecules were tested for binding to the opioid receptors and for analgesic effect delivered either to the brain (ICV) or to the bloodstream (IV). Table 2 is appended to the end of this specification.

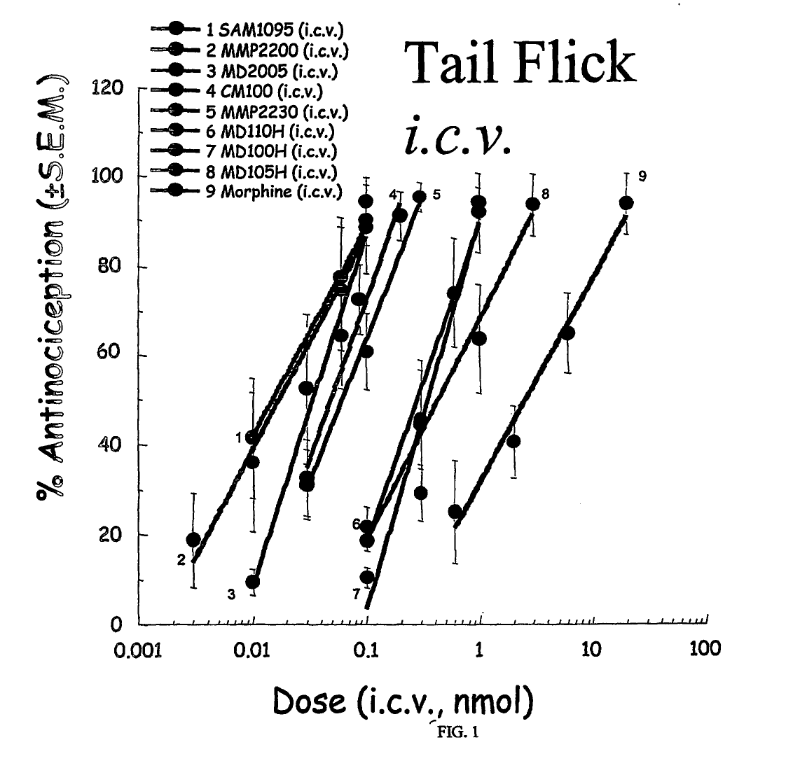

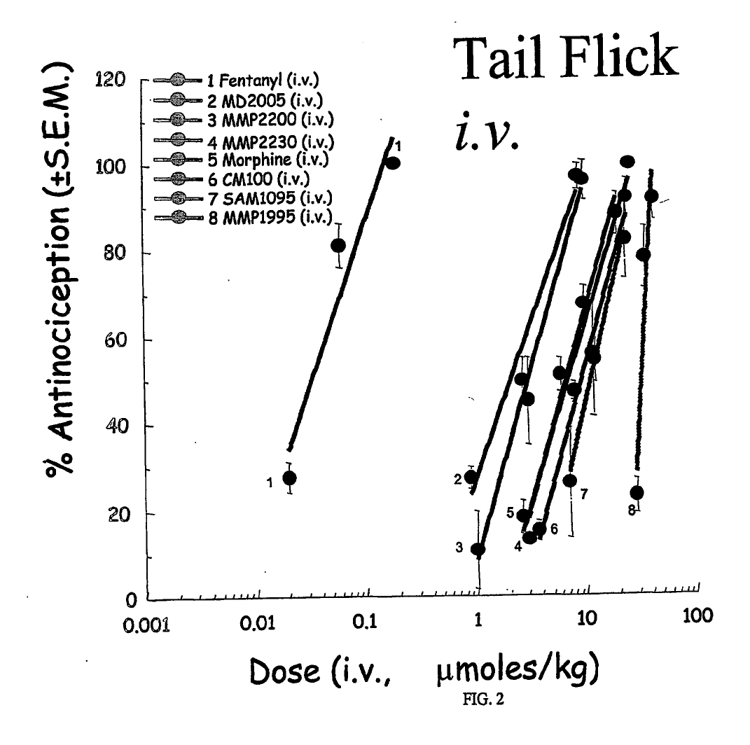

The data from the tail flick assays for anti-nociception was graphed and some of the illustrative data is presented in FIG. 1 and FIG. 2. Note in FIG. 1, where the molecules are delivered into the brain, i.e. i.c.v., that all of the tested molecules delivered effective analgesia at concentrations lower than that of morphine. In essence, in the brain, the molecules are all more effective than morphine in pain relief. Note, by comparison, that in FIG. 2, where the molecules were delivered to the bloodstream, i.e. i.v., that only some of the tested molecules produced antinociception that was more potent than morphine. Those molecules, designated here MMP2230, MMP2200, and MD2005, differ from the other molecules only in that they each have a disaccharide sugar attached. Note the comparison in particular to SAM1095 which has a monosaccharide group attached. In other words, while these enkephalin molecules are generally more potent than morphine when delivered to the brain, only the enkephalins with disaccharide sugars attached were effectively transferred across the blood-brain barrier in sufficient efficiencies to be more potent than morphine when delivered intravenously. The disaccharide glycopeptides have been given coined drug names based on the particular sugar moiety attached to the peptide, i.e. Maltomorphin (P2230), Lactomorphin (MMP 2200) and Biomorphin (MD 2005).

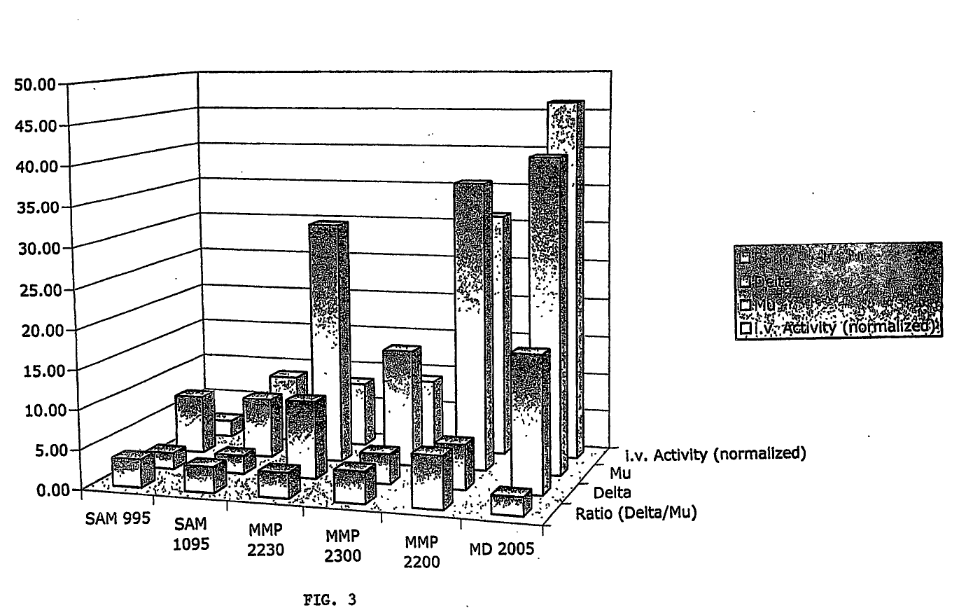

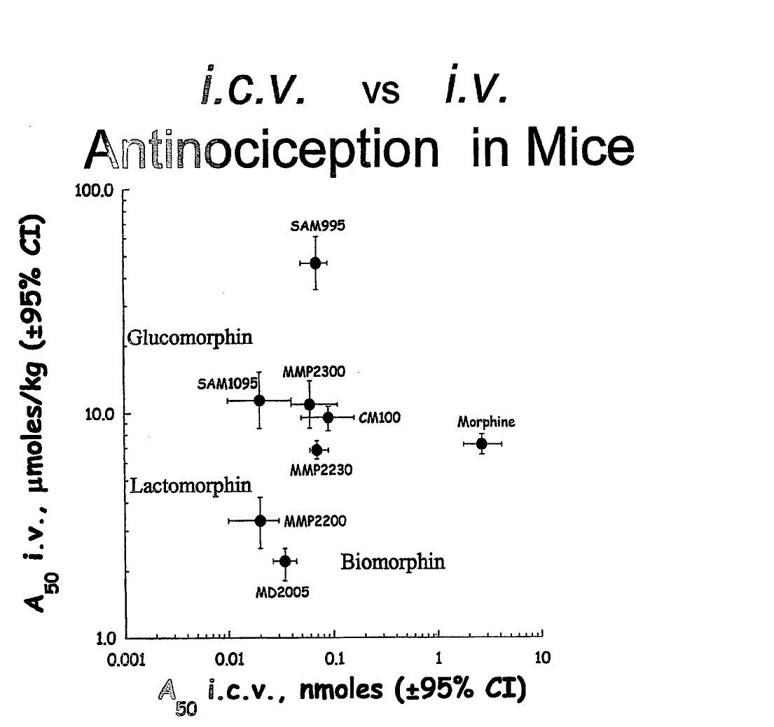

Two other charts also help to illustrate this phenomenon. In FIG. 3, a three dimensional bar graph shows the binding efficiencies to the opioid receptors and the normalized intravenous level of activity for several of the compounds on Table 2. Note the strong increase in the level of intravenous activity for several of the enkephalins with disaccharide sugars, again compared to similar peptides with either a monosaccharide (SAM1095) or a trisaccharide (MMP2300) attached. Similarly, in FIG. 4, effectiveness by i.v. administration is plotted against effectiveness by i.c.v. administration. Note that all of the glycopeptides with disaccharides attached fall on this chart below the best glycopeptide with a monosaccharide attached (SAM1095) and that two of the examples (MMP2200 and MD2005) are dramatically lower. These differences can only be explained by efficiency in transport across the blood-brain barrier, and explanation for that difference in transport lies with the nature of the sugar attached to the peptide.

Although as potent as morphine, it is believed that the glycosylated enkephalins of the class described here will have side effect profiles superior to opiates from plants. These molecules will produce less respiratory depression (which have observed), less constipation (based on the literature of delta opioid agonists) and have less active metabolites compared to morphine and other non-peptidic compounds.

It has also been found that these same drugs appear to have antidepressive properties. This property was tested in an animal model for depression, an animal model conventionally used to test drugs for possible antidepressive properties. In particular, the disaccharide glycopeptide MMP2200 (20 mg/kg, i.p., −30 minutes) was found to produce a significant decrease in immobility time compared to saline injected mice in the forced swim test. The control injected mice has a mean immobility time of about 85 seconds, while the mice injected with MMP2200 had a mean immobility time of less than 60 seconds. The results indicated that MMP2200 may have antidepressant activity, with efficacy similar to an SSRI such as fluoxetine and somewhat less efficacy compared to a tricylic such as desipramine.

TABLE 2
Receptor Binding Characteristics

| | | Address | Delta | MU | MVD | GPI | ICV (Mouse) | IV (Mouse) | +Err (95%) |
|---|---|---|---|---|---|---|---|---|---|
| ID Code | Message | (Transport Sequence) | nM | nM | nM | nM | IC50 nmol | μmol/kg | μmol/kg |
| Morphine | Morphine | Morphine | | | | | 2.7 | 6.3 | 4.9-7.9 |
| SAM 995 | YtGFL | S-CONH2 | 2.1 | 7.5 | 2.723 | 25.04 | 0.07 | 46.4 | 35.4-60.7 |
| SAM 1095 | YtGFL | L-Ser(b-Glc) CONH2 | 2.37 | 7.63 | 1.56 | 33.83 | 0.02 | 11.4 | 8.5-15.2 |
| MMP 2120 | YtGFL | L-Ser (a-Man) CONH2 | 22.95 | 15.2 | 3.029 | 23.25 | 0.04 | 31.6 | 26.5-37.8 |
| MMP 2200 | YtGFL | L-Ser (b-Lactose) CONH2 | 17.3 | 40 | 5.727 | 34.75 | 0.02 | 32 | 2.5-4.2 |
| MMP 2205 | YtGEL | L-Ser, L-Ser (b-Glc) CONH2 | | | 1.169 | 53.51 | 0.3 | 140.8 | 78-253.9 |
| MMP 2230 | YtGFL | L-Ser (b-Maltose) CONH2 | 9.86 | 30.8 | 1.705 | 52.57 | 0.07 | ˜12 | — |
| MMP 2300 | YtGFL | L-Ser (b-Maltotriose) CONH2 | 3.8 | 15 | 7.73 | 71.73 | 0.06 | 10.9 | 8.5-13.9 |
| CM 100 | YtGFL | L-Ser (b-Xyl) CONH2 | | | | | ˜0.04 | 9.45 | 8.34-10.71 |
| MD 2005 | YtGFL | L-Ser (b-Melbiose) CONH2 | 5.6 | 36.6 | | | 0.034 | 2.16 | 1.84-2.53 |
| MD 100H | YtGFL | PNLBEKALKS*L-CONH2 | | | | | ˜0.03 | | |
| MD 105H | YtGFL | (beta-Ala)NLBEKALKS*L-CONH2 | 47.3 | 62.1 | | | ˜0.03 | | |
| MD 110H | YtGFL | GGNLBEKALKS*L-CONH2 | 35 | 81 | | | ˜0.03 | | |

I/We claim:

1. A method for delivering analgesia to an individual comprising administering to the bloodstream of the individual an effective amount of an analgesic molecule which is a glycosylated enkephalin, the glycosylation being a disaccharide sugar moiety.

2. A method as claimed in claim 1 wherein the enkephalin includes the motif of Tyr-D-Thr-Gly-Phe.

3. A method as claimed in claim 1 wherein the glycosylated enkephalin is selected from the group consisting of the molecules designated MMP2200 and MMP2005 in Table 2.

4. A method for modifying a peptide enkephalin to enable the molecule to be transported across the blood-brain barrier, the method comprising the step of adding to the peptide a disaccharide moiety.

5. A compound of the formula X-O-G, wherein X is a peptide enkephalin which binds to an opioid receptor and G is a disaccharide sugar, the O-linkage of the peptide enkephalin to the disaccharide sugar being located on the peptide in the address region of the peptide.

6. A compound as claimed in claim 5 wherein the peptide comprises a message sequence selected from the group consisting of YGGF and YxGF, where x is a D-amino acid.

7. A pharmaceutical composition comprising a drug delivery package labeled for use as a human drug, the package containing a glycosylated enkephalin peptide, the glycosylation being a disaccharide attached to the message region of the peptide.

8. The glycosylated peptide compound YtGFLS(b-melibiose)CONH₂.

9. The glycosylated peptide compound YtGFLS(b-lactose)CONH₂ in solution and packaged for use as an injectable pharmaceutical.

10. The glycosylated peptide compound YtGFLS(b-maltose)CONH₂ in solution and packaged for use as an injectable pharmaceutical.
